(19) United States
(12) Patent Application Publication
(10) Pub. No.: US 2023/0114854 A1
(43) Pub. Date: Apr. 13, 2023

(54) CHIMERIC ANTIGEN RECEPTOR TARGETING B-CELL MATURATION ANTIGEN AND USE THEREOF

(71) Applicant: CELLGENTEK CO., LTD., Cheongju-si, Chungcheongbuk-do (KR)

(72) Inventors: Suk Gil SONG, Cheongju-si, Chungcheongbuk-do (KR); Hye Ran SUNG, Cheongju-si, Chungcheongbuk-do (KR); Seo Jin HWANG, Cheongju-si, Chungcheongbuk-do (KR)

(73) Assignee: CELLGENTEK CO., LTD., Cheongju-si, Chungcheongbuk-do (KR)

(21) Appl. No.: 17/904,057

(22) PCT Filed: Feb. 9, 2021
(86) PCT No.: PCT/KR2021/001681

§ 371 Date: Aug. 11, 2022

(30) Foreign Application Priority Data
Feb. 14, 2020 (KR) 10-2020-0018317

(51) Int. Cl. C07K 16/28; C12N 5/0783; A61P 35/00; C12N 15/85; C07K 14/705

(52) U.S. Cl. CPC C07K 16/2878; C12N 5/0646; A61P 35/00; C12N 15/85; C07K 14/70517; C07K 14/70521; C07K 14/70596; C07K 2319/03; C07K 2319/02

(57) ABSTRACT

Provided are a chimeric antigen receptor that targets B-cell maturation antigen (BCMA), and a use thereof, wherein immune cells expressing the chimeric antigen receptor can be effectively used in the treatment and prevention of B cell-related diseases, particularly multiple myeloma or non-Hodgkin's lymphoma.

### TECHNICAL FIELD

The present invention relates to a chimeric antigen receptor that targets B-cell maturation antigen (BCMA), and use thereof.

### BACKGROUND ART

Multiple myeloma or plasma cell myeloma is generally a cancer of the plasma cells, which is a type of white blood cell that is responsible for producing antibodies. In multiple myeloma patients, normal plasma cells transform to abnormal cancer cells, myeloma cells, and undergo rapid proliferation. Damaged myeloma cells produce gradually increasing amount of malignant myeloma cells in the bone marrow, and this process destroys the normal immune system in the body.

Treatment for multiple myeloma includes anticancer drug treatment such as melphalan, vincristine, doxorubicin, cyclophosphamide, steroid, bortezomib/velcade, thalidomide, and lenalidomide; radiation therapy; hematopoietic stem cell transplantation; and surgical interventions, and the like.

Chimeric antigen receptor T cells (CAR T cells) are T cells that have been genetically engineered to produce an artificial T cell receptor for use in immunotherapy. Chimeric antigen receptors (CAR) are receptor proteins that have been engineered to give T cells the new ability to target a specific protein. CARs are composed of an ectodomain, a transmembrane domain, and an endodomain, and depending on the composition of the endodomain transmitting an activation signal to cells, CARs have evolved from first generation, second generation, third generation, and fourth generation.

B cell maturation antigen (BCMA), which is a transmembrane glycoprotein that belongs to TNFRSF17 (tumor necrosis factor receptor superfamily 17), is expressed at a very high level in multiple myeloma (MM) cells in the patients. In addition, BCMA is expressed in various cancers involving B lymphocytes, such as malignant B cells, Hodgkin's lymphoma, and chronic lymphocytic leukemia.

BCMA is an antigen targeted by CAR T-cells that shows a significant therapeutic effect in clinical trials. Also recently, the first anti-BCMA antibody-drug conjugate tested in clinical trials produced a significant result in patients who do not respond well to medications such as anti-CD38 antibodies, proteosome inhibitors, and immunoregulators. However, presently, there has been no significant progress made in terms of research and development of NK cells having anti-BCMA CARs.

### PRIOR ART DOCUMENTS

### Patent Documents

PCT/US2015/041722
PCT/US2015/064269
PCT/IB2016/051808
PCT/CN2017/096938

### DISCLOSURE OF THE INVENTION

### Technical Problem

An aspect of the present invention provides a chimeric antigen receptor (CAR) that specifically recognizes BCMA, anti-BCMA CAR.

Another aspect of the present invention provides a nucleic acid molecule encoding the anti-BCMA CAR, and a vector including the nucleic acid molecule.

Another aspect of the present invention provides a cell expressing the anti-BCMA CAR by including the nucleic acid molecule or the vector.

Another aspect of the present invention provides a composition comprising the cell expressing the anti-BCMA CAR.

Another aspect of the present invention provides a pharmaceutical composition for treating or preventing B cell-related conditions, comprising the cell expressing the anti-BCMA CAR.

Another aspect of the present invention provides a therapeutic use of the cell expressing the anti-BCMA CAR for B cell-related conditions.

Another aspect of the present invention provides a method of treating B cell-related conditions by using the cell expressing the anti-BCMA CAR.

### Technical Solution

According to an aspect of the present invention, provided is a chimeric antigen receptor (CAR) comprising the following polypeptides:

(i) an extracellular antigen binding domain that specifically binds to a B cell maturation antigen (BCMA);

(ii) a transmembrane domain;

(iii) an intracellular co-stimulatory signaling domain; and

(iv) an intracellular primary signaling domain.

According to another aspect of the present invention, provided is a nucleic acid molecule encoding the CAR.

According to another aspect of the present invention, provided is a vector comprising the polynucleotide.

According to another aspect of the present invention, provided is a cell comprising the nucleic acid molecule or the vector.

According to another aspect of the present invention, provided is a pharmaceutical composition for treating or preventing B cell-related conditions, comprising a pharmaceutically effective amount of the cell including the vector.

### Advantageous Effects

Immune cells expressing the chimeric antigen receptor of the present invention may be effectively used for the treatment and prevention of B cell-related conditions, in particular, multiple myeloma or non-Hodgkin's lymphoma.

### BRIEF DESCRIPTION OF THE DRAWINGS

FIG. 1A shows a BCMA-chimeric antigen receptor (CAR) construct according to one embodiment of the present invention. SP is a signal peptide, TM is a transmembrane domain, CS1-ICD is a co-stimulatory 1-intracellular signaling domain; and CS2-ICD is a co-stimulatory 2-intracellular signaling domain.

FIG. 1B is a schematic diagram more specifically showing a BCMA-CAR construct according to one example of the present invention.

FIG. 2 shows the expression of BCMA-CAR introduced into NK cells as measured by Western blotting using anti-phospho-CD3ζ antibodies. CGT301, CGT302, CGT303, and CGT304 are NK cells transfected with BCMA-CAR1, BCMA-CAR2, BCMA-CAR3, and BCMA-CAR4 of the present invention, respectively.

FIG. 3 shows BCMA-CAR expression on the surface of NK cells, as measured by flow cytometry using human IgG(ab′)2 antibodies, and His tag conjugated-BCMA recombinant protein and PE-conjugated His antibodies.

FIG. 4A shows a measurement result of cytolytic activity of BCMA-CAR NK cells toward cancer cells, FIG. 4B shows a measurement result of the amount of IFNγ released from BCMA-CAR NK cells, and FIG. 4C shows a measurement result of the amount of Granzyme B released from BCMA-CAR NK cells.

FIG. 5 shows BCMA expression observed in K562 cancer cells lacking BCMA expression, after transduced with BCMA gene derived from human (h), monkey (Rh), mouse (Ms), or rat (Rat), respectively.

FIG. 6A and FIG. 6B show antigen specificity of BCMA-CAR of the present invention, observed with respect to BCMA derived from human (hu1, hu2), monkey (Rh), mouse (Ms), and rat (Rat).

FIG. 7 shows the result of a competition assay using rhBCMA proteins, performed to determine whether activity of BCMA-CAR NK cells is a BCMA-specific response.

FIG. 8 shows the result of a competition assay using BCMA mAb, performed to determine whether activity of BCMA-CAR NK cells is a BCMA-specific response.

FIG. 9 shows a measurement result of in vivo anticancer efficacy of BCMA-CAR NK cells.

FIG. 10A to FIG. 10D show the result of measuring stability of BCMA-CAR NK cells, and in particular, FIG. 10A shows a result of cell viability measurement, FIG. 10B shows a result of population doubling time measurement, FIG. 10C shows a result of BCMA-CAR expression measurement, and FIG. 10D shows a result of cytotoxicity measurement.

### MODE FOR CARRYING OUT THE INVENTION

In one aspect of the present invention, the present invention provides a chimeric antigen receptor (CAR) including the following polypeptides:

(i) an extracellular antigen binding domain that specifically binds to B cell maturation antigen (BCMA);

(ii) a transmembrane domain;

(iii) an intracellular co-stimulatory signaling domain; and

(iv) an intracellular primary signaling domain.

In one embodiment, the extracellular antigen binding domain comprises an anti-BCMA antibody or antigen binding fragment thereof. In particular, the antigen binding fragment may be selected from the group consisting of Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, nanobody).

In one embodiment, the extracellular antigen binding domain comprises (i) a light chain variable region (V_{L}) of the anti-BCMA antibody, comprising L-CDR (complementarity determining region)1, L-CDR2, and L-CDR3;

(ii) a heavy chain variable region (V_{H}) of the anti-BCMA antibody, comprising H-CDR1, H-CDR2, and H-CDR3; or

(iii) at least one of the light chain variable region, and at least one of the heavy chain variable region.

The term “specifically binds” as used herein refers to forming of a relatively stable complex by an antibody or antigen binding fragment thereof, or a construct e.g., scFv, with an antigen, under the physiological conditions.

The term “variable region” as used herein refers to a domain of an antibody heavy chain or light chain that is involved in binding of an antibody to an antigen. In a native antibody, heavy chain variable region (V_{H}) and light chain variable region (V_{L}) generally have a similar structure, wherein each domain comprises 4 conserved framework regions (FRs) and 3 hypervariable regions (HVRs). (Kindt et al., Kuby Immunology, Sixth Edition, W.H. Freeman and Co., page 91 (2007)).

The term “CDR (complementarity determining region)” as used herein refers to the amino acid sequence of a hypervariable region of immunoglobulin heavy chain and light chain (Kabat et al., Sequences of Proteins of Immunological Interest, 4th Ed., U.S. Department of Health and Human Services, National Institutes of Health (1987)).

Heavy chain variable region (V_{H}) and light chain variable region (V_{L}) each comprise 3 CDRs (heavy chain: H-CDR1, H-CDR2, and H-CDR3; and light chain: L-CDR1, L-CDR2, and L-CDR3). Such CDRs provide main contact residues for binding an antibody to an antigen or an epitope.

In one embodiment, the extracellular antigen binding domain may comprise at least one light chain variable region (V_{L}), or at least one heavy chain variable region (V_{H}), and for example, may comprise a structure of (V_{L}), (V_{L})-(V_{L}), (V_{L})-(V_{L})-(V_{L}), (V_{L})-(V_{L})-(V_{L})-(V_{L}), (V_{L})-(V_{L})-(V_{L})-(V_{L})-(V_{L}), or (V_{L})-(V_{L})-(V_{L})-(V_{L})-(V_{L})-(V_{L}), and alternatively may comprise a structure of (V_{H}), (V_{H})-(V_{H}), (V_{H})-(V_{H})-(V_{H}), (V_{H})-(V_{H})-(V_{H})-(V_{H}), (V_{H})-(V_{H})-(V_{H})-(V_{H})-(V_{H}), or (V_{H})-(V_{H})-(V_{H})-(V_{H})-(V_{H})-(V_{H}).

In one embodiment, the extracellular antigen binding domain may comprise a structure in which one light chain variable region (V_{L}) and one heavy chain variable region (V_{H}) are connected, and may comprise the structure in one orientation (V_{L})-(V_{H}), or in the other orientation (V_{H})-(V_{L}).

In one embodiment, the extracellular antigen binding domain may comprise a structure in which one, or two or more heavy chain variable regions (V_{H}) are connected to two or more light chain variable regions (V_{L}); or a structure in which two or more heavy chain variable regions (V_{H}) are connected to one, or two or more light chain variable regions (V_{L}).

For example, the extracellular antigen binding domain may comprise a structure of (V_{L})-(V_{L})-(V_{H}), (V_{L})-(V_{L})-(V_{H})-(V_{H}), (V_{L})-(V_{L})-(V_{H})-(V_{H})-(V_{H}), (V_{L})-(V_{L})-(V_{H})-(V_{H})-(V_{H})-(V_{H}), (V_{L})-(V_{L})-(V_{L})-(V_{H}), (V_{L})-(V_{L})-(V_{L})-(V_{H})-(V_{H}), (V_{L})-(V_{L})-(V_{L})-(V_{H})-(V_{H})-(V_{H}), (V_{L})-(V_{H})-(V_{H}), or (V_{L})-(V_{H})-(V_{H})-(V_{H}).

In one embodiment, the extracellular antigen binding domain may comprise a structure in which two or more light chain variable regions are connected to one, or two or more heavy chain variable regions; or a structure in which two or more heavy chain variable regions are connected to one, or two or more light chain variable regions. For example, the extracellular antigen binding domain may comprise a structure of (V_{H})-(V_{H})-(V_{L}), (V_{H})-(V_{H})-(V_{L})-(V_{L}), (V_{H})-(V_{H})-(V_{L})-(V_{L})-(V_{L}), (V_{H})-(V_{H})-(V_{L})-(V_{L})-(V_{L})-(V_{L}), (V_{H})-(V_{H})-(V_{H})-(V_{L}), (V_{H})-(V_{H})-(V_{H})-(V_{L})-(V_{L}), (V_{H})-(V_{H})-(V_{H})-(V_{L})-(V_{L})-(V_{L}), (V_{L})-(V_{H})-(V_{H}), (V_{L})-(V_{L})-(V_{H})-(V_{H}), (V_{L})-(V_{L})-(V_{L})-(V_{H})-(V_{H}), (V_{L})-(V_{L})-(V_{L})-(V_{L})-(V_{H})-(V_{H}), or (V_{L})-(V_{L})-(V_{L})-(V_{L})-(V_{H})-(V_{H})-(V_{H}).

In one embodiment, the extracellular antigen binding domain may comprise a structure in which the heavy chain variable region (V_{H}) and the light chain variable region (V_{L}) are alternately connected. For example, the extracellular antigen binding domain may comprise a structure of (V_{H})-(V_{L})-(V_{H}), (V_{H})-(V_{L})-(V_{H})-(V_{L}), (V_{H})-(V_{L})-(V_{H})-(V_{L})-(V_{H}), (V_{L})-(V_{H})-(V_{L}), (V_{L})-(V_{H})-(V_{L})-(V_{H}), or (V_{L})-(V_{H})-(V_{L})-(V_{H})-(V_{L}).

In one embodiment,

the L-CDR1 is a polypeptide having the amino acid sequence of SEQ ID NO: 1, SEQ ID NO: 7, SEQ ID NO: 13, or SEQ ID NO: 19;

the L-CDR2 is a polypeptide having the amino acid sequence of SEQ ID NO: 2, SEQ ID NO: 8, SEQ ID NO: 14, or SEQ ID NO: 20;

the L-CDR3 is a polypeptide having the amino acid sequence of SEQ ID NO: 3, SEQ ID NO: 9, SEQ ID NO: 15, or SEQ ID NO: 21;

the H-CDR1 is a polypeptide having the amino acid sequence of SEQ ID NO: 4, SEQ ID NO: 10, SEQ ID NO: 16, or SEQ ID NO: 22;

the H-CDR2 is a polypeptide having the amino acid sequence of SEQ ID NO: 5, SEQ ID NO: 11, SEQ ID NO: 17, or SEQ ID NO: 23; and

the H-CDR3 is a polypeptide having the amino acid sequence of SEQ ID NO: 6, SEQ ID NO: 12, SEQ ID NO: 18, or SEQ ID NO: 24.

In one embodiment, the extracellular antigen binding domain may comprise: a heavy chain variable region (V_{H}) of the anti-BCMA antibody, including H-CDR1 having the amino acid sequence of SEQ ID NO: 4, H-CDR2 having the amino acid sequence of SEQ ID NO: 5, and H-CDR3 having the amino acid sequence of SEQ ID NO: 6; and a light chain variable region (V_{L}) of the anti-BCMA antibody, including L-CDR1 having the amino acid sequence of SEQ ID NO: 1, L-CDR2 having the amino acid sequence of SEQ ID NO: 2, and L-CDR3 having the amino acid sequence of SEQ ID NO: 3.

In one embodiment, the extracellular antigen binding domain may comprise: a heavy chain variable region (V_{H}) of the anti-BCMA antibody, including H-CDR1 having the amino acid sequence of SEQ ID NO: 10, H-CDR2 having the amino acid sequence of SEQ ID NO: 11, and H-CDR3 having the amino acid sequence of SEQ ID NO: 12; and a light chain variable region (V_{L}) of the anti-BCMA antibody, including L-CDR1 having the amino acid sequence of SEQ ID NO: 7, L-CDR2 having the amino acid sequence of SEQ ID NO: 8, and L-CDR3 having the amino acid sequence of SEQ ID NO: 9.

In one embodiment, the extracellular antigen binding domain may comprise: a heavy chain variable region (V_{H}) of the anti-BCMA antibody, including H-CDR1 having the amino acid sequence of SEQ ID NO: 16, H-CDR2 having the amino acid sequence of SEQ ID NO: 17, and H-CDR3 having the amino acid sequence of SEQ ID NO: 18; and a light chain variable region (V_{L}) of the anti-BCMA antibody, including L-CDR1 having the amino acid sequence of SEQ ID NO: 13, L-CDR2 having the amino acid sequence of SEQ ID NO: 14, and L-CDR3 having the amino acid sequence of SEQ ID NO: 15.

In one embodiment, the extracellular antigen binding domain may comprise: a heavy chain variable region (V_{H}) of the anti-BCMA antibody, including H-CDR1 having the amino acid sequence of SEQ ID NO: 22, H-CDR2 having the amino acid sequence of SEQ ID NO: 23, and H-CDR3 having the amino acid sequence of SEQ ID NO: 24; and a light chain variable region (V_{L}) of the anti-BCMA antibody, including L-CDR1 having the amino acid sequence of SEQ ID NO: 19, L-CDR2 having the amino acid sequence of SEQ ID NO: 20, and L-CDR3 having the amino acid sequence of SEQ ID NO: 21.

In one embodiment, the extracellular antigen binding domain comprises one, or two or more from among

(i) at least one light chain variable region (V_{L}) of the anti-BCMA antibody, selected from the group consisting of polypeptides having the amino acid sequences of SEQ ID NO: 25, SEQ ID NO: 27, SEQ ID NO: 29, and SEQ ID NO: 31; and

(ii) at least one heavy chain variable region (V_{H}) of the anti-BCMA antibody, selected from the group consisting of polypeptides having the amino acid sequences of SEQ ID NO: 26, SEQ ID NO: 28, SEQ ID NO: 30, and SEQ ID NO: 32.

Combinations of the light chain variable region (V_{L}) and the heavy chain variable region (V_{H}) in the present invention are as discussed above.

In particular, the extracellular antigen binding domain may comprise at least one light chain variable region (V_{L}), or at least one heavy chain variable region (V_{H}), and for example, may comprise a structure of (V_{L}), (V_{L})-(V_{L}), (V_{L})-(V_{L})-(V_{L}), (V_{L})-(V_{L})-(V_{L})-(V_{L}), (V_{L})-(V_{L})-(V_{L})-(V_{L})-(V_{L}), or (V_{L})-(V_{L})-(V_{L})-(V_{L})-(V_{L})-(V_{L}), and alternatively may comprise a structure of (V_{H}), (V_{H})-(V_{H}), (V_{H})-(V_{H})-(V_{H}), (V_{H})-(V_{H})-(V_{H})-(V_{H}), (V_{H})-(V_{H})-(V_{H})-(V_{H})-(V_{H}), or (V_{H})-(V_{H})-(V_{H})-(V_{H})-(V_{H})-(V_{H}).

In addition, the extracellular antigen binding domain may comprise a structure in which one light chain variable region (V_{L}) and one heavy chain variable region (V_{H}) are connected, and may comprise the structure in one orientation (V_{L})-(V_{H}), or in the other orientation (V_{H})-(V_{L}).

In addition, the extracellular antigen binding domain may comprise a structure of (V_{L})-(V_{L})-(V_{H}). (V_{L})-(V_{L})-(V_{H})-(V_{H}), (V_{L})-(V_{L})-(V_{H})-(V_{H})-(V_{H}) (V_{L})-(V_{L})-(V_{H})-(V_{H})-(V_{H})-(V_{H}), (V_{L})-(V_{L})-(V_{L})-(V_{H}), (V_{L})-(V_{L})-(V_{L})-(V_{H})-(V_{H}), (V_{L})-(V_{L})-(V_{L})-(V_{H})-(V_{H})-(V_{H}), (V_{L})-(V_{H})-(V_{H}), (V_{L})-(V_{H})-(V_{H})-(V_{H}), or (V_{L})-(V_{H})-(V_{H})-(V_{H})-(V_{H}).

In addition, the extracellular antigen binding domain may comprise a structure of (V_{H})-(V_{H})-(V_{L}), (V_{H})-(V_{H})-(V_{L})-(V_{L}), (V_{H})-(V_{H})-(V_{L})-(V_{L})-(V_{L}), (V_{H})-(V_{H})-(V_{L})-(V_{L})-(V_{L})-(V_{L}), (V_{H})-(V_{H})-(V_{H})-(V_{L}), (V_{H})-(V_{H})-(V_{H})-(V_{L})-(V_{L}), (V_{L})-(V_{H})-(V_{H}), (V_{L})-(V_{L})-(V_{H})-(V_{H}), (V_{L})-(V_{L})-(V_{L})-(V_{H})-(V_{H}), (V_{L})-(V_{L})-(V_{L})-(V_{L})-(V_{H})-(V_{H}), or (V_{L})-(V_{L})-(V_{L})-(V_{L})-(V_{H})-(V_{H})-(V_{H}).

In one embodiment, the extracellular antigen binding domain may comprise a structure in which a heavy chain variable region (V_{H}) and a light chain variable region (V_{L}) are alternately connected. For example, the extracellular antigen binding domain may comprise a structure of (V_{H})-(V_{L})-(V_{H}), (V_{H})-(V_{L})-(V_{H})-(V_{L}), (V_{H})-(V_{L})-(V_{H})-(V_{L})-(V_{H}), (V_{L})-(V_{H})-(V_{L}), (V_{L})-(V_{H})-(V_{L})-(V_{H}), or (V_{L})-(V_{H})-(V_{L})-(V_{H})-(V_{L}).

In one embodiment, the extracellular antigen binding domain may comprise a heavy chain variable region (V_{H}) of the anti-BCMA antibody, having the amino acid sequence of SEQ ID NO: 26; and a light chain variable region (V_{L}) of the anti-BCMA antibody, having the amino acid sequence of SEQ ID NO: 25.

In one embodiment, the extracellular antigen binding domain may comprise a heavy chain variable region (V_{H}) of the anti-BCMA antibody, having the amino acid sequence of SEQ ID NO: 28; and a light chain variable region (V_{L}) of the anti-BCMA antibody, having the amino acid sequence of SEQ ID NO: 27.

In one embodiment, the extracellular antigen binding domain may comprise a heavy chain variable region (V_{H}) of the anti-BCMA antibody, having the amino acid sequence of SEQ ID NO: 30; and a light chain variable region (V_{L}) of the anti-BCMA antibody, having the amino acid sequence of SEQ ID NO: 29.

In one embodiment, the extracellular antigen binding domain may comprise a heavy chain variable region (V_{H}) of the anti-BCMA antibody, having the amino acid sequence of SEQ ID NO: 32; and a light chain variable region (V_{L}) of the anti-BCMA antibody, having the amino acid sequence of SEQ ID NO: 31.

In one embodiment, the chimeric antigen receptor of the present invention may further comprise a linker positioned between two or more light chain or heavy chain variable regions.

In one embodiment, the linker may be a polypeptide having the amino acid sequence of SEQ ID NO: 37 or SEQ ID NO: 38.

In one embodiment, the extracellular antigen binding domain of the present invention may further comprise a hinge region connected to the transmembrane domain.

In one embodiment, the hinge region may comprise an IgG1 hinge region, an IgG4 hinge region, or a CD8α hinge region.

In one embodiment, the IgG1 hinge region comprises a polypeptide having the amino acid sequence of SEQ ID NO: 39; the IgG4 hinge region comprises a polypeptide having the amino acid sequence of SEQ ID NO: 40; and the CD8α hinge region comprises a polypeptide having the amino acid sequence of SEQ ID NO: 41.

In one embodiment, the transmembrane domain comprises a CD8α transmembrane region or a CD28 transmembrane region.

In one embodiment, the CD8α transmembrane region comprises a polypeptide having the amino acid sequence of SEQ ID NO: 42, and the CD28 transmembrane region comprises a polypeptide having the amino acid sequence of SEQ ID NO: 43.

In one embodiment, the intracellular co-stimulatory signaling domain comprises an intracellular co-stimulatory signaling domain of CD28, DAP10, or CD137(4-1BB).

In one embodiment, the intracellular co-stimulatory signaling domain of CD28 comprises a polypeptide having the amino acid sequence of SEQ ID NO: 44.

In one embodiment, the intracellular co-stimulatory signaling domain of DAP10 comprises a polypeptide having the amino acid sequence of SEQ ID NO: 45.

In one embodiment, the intracellular co-stimulatory signaling domain of CD137(4-1BB) comprises a polypeptide having the amino acid sequence of SEQ ID NO: 46.

In one embodiment, the intracellular primary signaling domain comprises an intracellular domain of CD3ζ.

In one embodiment, the intracellular domain of CD3ζ comprises a polypeptide having the amino acid sequence of SEQ ID NO: 47.

In one embodiment, the intracellular co-stimulatory signaling domain and/or the intracellular primary signaling domain may be connected via a linker.

In one embodiment, the linker connecting the intracellular co-stimulatory signaling domain and/or the intracellular primary signaling domain is a polypeptide having the amino acid sequence of SEQ ID NO: 38.

In one embodiment, the extracellular antigen binding domain further comprises a signal peptide.

In one embodiment, the signal peptide comprises a signal peptide of CD16, GM-CSF, human IgG, or CD8.

In one embodiment, the signal peptide of CD16 comprises a polypeptide having the amino acid sequence of SEQ ID NO: 33; the signal peptide of GM-CSF comprises a polypeptide having the amino acid sequence of SEQ ID NO: 34; the signal peptide of human IgG comprises a polypeptide having the amino acid sequence of SEQ ID NO: 35; and the signal peptide of CD8 comprises a polypeptide having the amino acid sequence of SEQ ID NO: 36.

In one embodiment, the chimeric antigen receptor of the present invention may comprise the following components described above: the heavy chain variable region (V_{H}) and light chain variable region (V_{L}) of the anti-BCMA antibody; the CD8α hinge region; the CD28 transmembrane region; the co-stimulatory signaling domain of CD137(4-1BB); and the intracellular domain of CD3ζ.

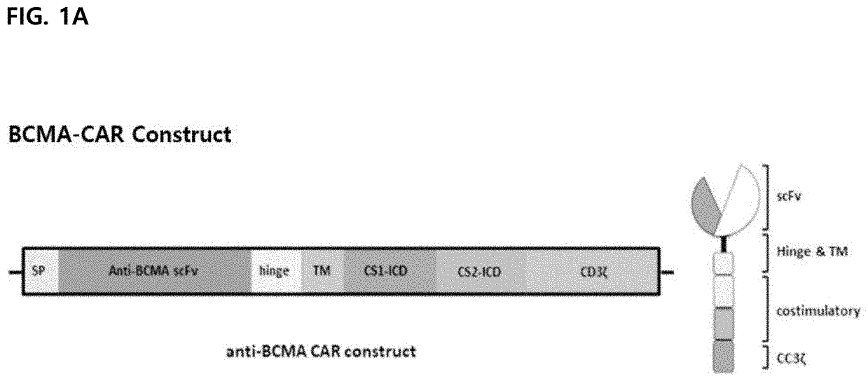

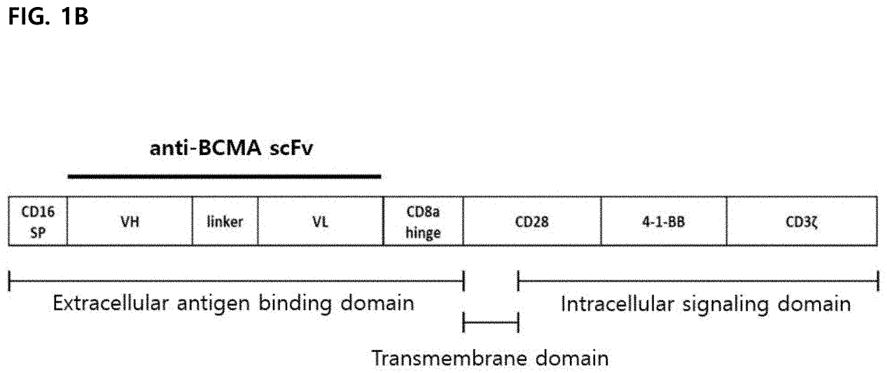

With reference to the following examples of the present invention, a more specific composition of polypeptides constituting the chimeric antigen receptor of the present invention may be expressed by FIG. 1B, but the chimeric antigen receptor of the present invention is not limited to the features in FIG. 1B.

In another aspect of the present invention, the present invention provides a nucleic acid molecule encoding the chimeric antigen receptor described above.

The term “nucleic acid molecule” as used herein refers to a polynucleotide, including DNA and RNA, wherein the DNA includes gDNA (genomic DNA) and cDNA (complementary DNA), and the RNA includes mRNA (messenger RNA). The mRNA is a nucleic acid molecule capable of directing translation of the chimeric antigen receptor of the present invention, and may include primary transcript mRNA molecules or mature mRNA molecules. Nucleotides as a basic constituent unit of the nucleic acid molecule include naturally occurring nucleotides, and also analogues having modified sugar or base moieties (Scheit, Nucleotide Analogs, John Wiley, New York (1980); Uhlman, and Peyman, Chemical Reviews, 90:543-584 (1990)).

Nucleic acid molecules or polynucleotides of the present invention may be obtained by chemical synthesis, recombinant methods, or PCR. Since methods of chemical synthesis of nucleic acid molecules are widely known in the art, one skilled in the art would be able to easily synthesize a nucleic acid molecule having a desired sequence, by using the sequences provided herein and a commercial nucleic acid molecule synthesis system.

In another aspect of the present invention, the present invention provides a vector including the nucleic acid molecule.

The vector may be a cloning vector, or an expression vector stably expressing the chimeric antigen receptor.

The vector may include an origin of replication and may include, without limitation, an origin of replication operable in eukaryotic cells, such as f1 origin of replication, SV origin of replication, pMB1 origin of replication, Adeno origin of replication, AAV origin of replication, BBV origin of replication, and the like. The vector includes an antibiotic-resistant gene commonly used as a selectable marker in the art, and for example, may include genes resistant to ampicillin, gentamicin, carbenicillin, chloramphenicol, streptomycin, kanamycin, geneticin, neomycin, puromycin, and tetracycline.

The nucleic acid molecule encoding the above-described chimeric antigen receptor in the vector of the present invention may be operably linked to a promoter sequence initiating the expression thereof. The promotor may include a promoter originated from the genome of a mammalian cell (e.g., metallothionine promoter, EF-1 alpha promoter) or a promoter originated from a mammalian virus (e.g., adenovirus late promoter, vaccinia virus 7.5K promoter, SV40 promoter, cytomegalovirus (CMV) promoter, and tk promoter of HSV), and may include a polyadenylated sequence as a transcription terminator sequence, e.g., SV40 polA sequence, BGH polA sequence, and the like.

In one embodiment, the vector or recombinant vector is a transposon vector, an episome vector, or a viral vector.

In another embodiment, the viral vector is a retroviral vector or a lentiviral vector.

In some embodiments, the lentiviral vector is selected from the group consisting essentially of: human immunodeficiency virus 1 (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus (SIV).

In another aspect of the present invention, the present invention provides a cell including the nucleic acid molecule, or a cell including the above-described vector.

In the present invention, a vector may be introduced into a cell. The vector may be introduced into the cell by transfection methods known in the art, and such methods include, but are not limited to, microinjection (Capecchi, M. R., Cell 22, 479 (1980)), calcium phosphate precipitation (Graham, F. L. et al., Virology 52, 456 (1973)), electroporation (Neumann, E. et al., EMBO J. 1, 841 (1982)), liposome-mediated transfection (Wong, T. K. et al., Gene, 10, 87 (1980)), DEAE-dextran treatment (Gopal, Mol. Cell Biol. 5, 1188-1190 (1985)), and gene bombardment (Yang et al., Proc. Natl. Acad. Sci. USA 87, 9568-9572 (1990)), and the like.

A vector introduced into a cell may remain as a non-integrating vector (e.g., plasmid) or may be integrated into a part of the chromosome DNA of the host cell.

A cell including a vector including a nucleic acid molecule (polynucleotide) encoding the above-described chimeric antigen receptor of the present invention stably expresses the chimeric antigen receptor inside the cell, and the expressed chimeric antigen receptor is stably situated at the cell membrane.

In one embodiment, a cell including a nucleic acid molecule (e.g., cDNA or mRNA molecule) encoding the chimeric antigen receptor of the present invention may be provided.

A cell including cDNA capable of directing transcription and translation of the chimeric antigen receptor of the present invention may stably express the chimeric antigen receptor inside the cell.

A mature mRNA or a primary transcript mRNA capable of directing translation of the chimeric antigen receptor of the present invention may be directly introduced into a cell to induce intracellular expression of the chimeric antigen receptor.

In one embodiment, the cell into which the vector is introduced is an immune cell, and preferably may be an NK (natural killer) cell, a T cell, a cytotoxic T cell, or a regulatory T cell. Preferably, the cell may be a human-derived immune cell, more preferably, a human-derived NK cell.

In the present invention, “T cell” refers to the type of lymphocyte that matures in the thymus. T cells play a critical role in cell-mediated immunity and are distinguished from other lymphocytes such as B lymphocytes by the presence of T-cell receptors on the cell surface. T cells may be isolated, or obtained from a commercially available source. T cells comprise immune cells of any kind that expresses CD3, including helper T cells (CD4+ cells), cytotoxic T cells (CD8+ cells), natural killer T cells, regulatory T cells (Treg), and gamma-delta T cells. “Cytotoxic cells” comprise CD8+ T cells, NK (natural killer) cells, and neutrophils, capable of mediating a cytotoxic response.

In the present invention, “NK cells”, also known as natural killer cells, are a type of lymphocyte derived from the bone marrow that plays a critical role in the innate immune system. Without the major histocompatibility complex or antigens on the cell surface, NK cells provide rapid immune responses with respect to virus-infected cells, tumor cells, or other stressed cells. Non-limiting examples of commercial NK cell lines comprise NK-92 (ATCC® CRL-2407™) and NK-92MI (ATCC® CRL-2408™). Additional examples include NK cell lines HANK1, KHYG-1, NKL, NK-YS, NOI-90, YT and NK101, but are not limited thereto. Non-limiting example of suppliers of such commercially available cell lines include American Type Culture Collection, or ATCC (http://www.atcc.org/), and the German Collection of Microorganisms and Cell Cultures (https://www.dsmz.de/).http://www.atcc.org/https://www.dsmz.de/

In another aspect of the present invention, the present invention provides a pharmaceutical composition for treating or preventing B cell-related conditions, including: (i) a pharmaceutically effective amount of a cell including a nucleic acid molecule encoding the above-described CAR, or a cell including a vector including the nucleic acid molecule; and (ii) a pharmaceutically acceptable carrier.

As used herein, the term “pharmaceutical composition” as used herein refers to a composition formulated in a pharmaceutically acceptable or physiologically acceptable solution for administration to cells or animals, either singly or in combination with one or more other therapeutic agents.

As used herein, the term “pharmaceutically effective amount” refers to “an amount effective” or “an effective amount” of a genetically modified therapeutic cell, e.g., NK cell, to achieve a beneficial or desired prophylactic or therapeutic result, including clinical results.

As used herein, the term “pharmaceutically acceptable carrier, diluent or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Examples of such pharmaceutically acceptable carriers include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

The phrase “pharmaceutically acceptable” refers to not giving rise to allergic response or other similar complications when administered to a human being or an animal. Such carriers include certain solvents, dispersion media, coating agents, antibacterial agents and antifungal agents, isotonic agents, absorption delaying agents, and the like. The use of such media and agents for pharmaceutical active substances is well known in the art.

The pharmaceutical composition of the present invention may be administered preferably via parenteral, intraperitoneal, intradermal, intramuscular, and intravenous routes.

The pharmaceutical composition of the present invention is administered in a therapeutically effective amount in a manner compatible with its formulation. In addition, the administration dose may be adjusted according to a state or condition of a subject to be treated. For parenteral administration of a water-based injection solution, the solution should be appropriately buffered according to necessity, and first, liquid diluent is made isotonic with sufficient saline or glucose. Such special water-based solutions are particularly appropriate for intravenous, intramuscular, subcutaneous, intradermal and intraperitoneal administrations.

Carriers, preparations, and media usable in the pharmaceutical composition of the present invention are known in the art (see “Remington's Pharmaceutical Sciences”, 1995, 15th Edition).

In one embodiment, the B cell related condition to be treated by the composition of the present invention is multiple myeloma, non-Hodgkin's lymphoma, B cell proliferation of uncertain malignant potential, lymphomatoid granulomatosis, posttransplant lymphoproliferative disorder, immunoregulatory disorders, rheumatoid arthritis, myasthenia gravis, idiopathic thrombocytopenic purpura, antiphospholipid syndrome, Chagas' disease, Grave's disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia, and rapidly progressive glomerulonephritis, heavy chain disease, primary or immune cell-associated amyloidosis, or monoclonal gammopathy of undetermined significance.

In one embodiment, the B cell-related condition is a B cell malignancy.

In one embodiment, the B cell malignancy is multiple myeloma or non-Hodgkins' lymphoma.

The specific examples described hereinbelow are for the purpose of describing preferred embodiments or examples of the present invention only, and thus should not be construed as limiting the scope of the invention. It will be apparent to those skilled in the art that modifications and other uses of the present invention do not depart from the scope of the invention described in the claims.

### EXAMPLES

### Example 1. Construction of BCMA-CAR

Design of BCNA-CAR

A third-generation chimeric antigen receptor (CAR) construct containing a scFv region derived from monoclonal antibodies specifically binding to BCMA was designed (FIG. 1).

The CAR of the present invention is a third-generation chimeric antigen receptor (CAR) composed of (i) a signal peptide; (ii) a BCMA recognition and binding domain; (iii) a hinge region; (iv) a transmembrane domain; (v) a CD3ζ signaling domain; and (vi) two co-stimulatory domains.

The amino acid sequences of each domain or polypeptide that may constitute the CAR are shown in Table 1.

TABLE 1

| | | SEQ |
|---|---|---|
| | | ID |
| SEQUENCE INFORMATION | Description | NO |
| MWQLLLPTALLLLVSA | CD16 signal peptide | 33 |
| MWLQSLLLLGTVACSIS | GM-CSF signal peptide | 34 |
| MDWTWRILFLVAAATGAHS | Human IgG signal peptide | 35 |
| MALPVTALLLPLALLLHAARP | CD8 signal peptide | 36 |
| QSVLTQPPSASGTPGQRVTISCSGSSSNIGSNSV | VL of scFv | 25 |
| SWYQQLPGTAPKLLIYADSKRPSGVPDRFSGSKS | (Light chain variable | |
| GTSASLAISGLRSEDEADYYCGSWDYSLSGYVFG | region 1) | |
| GGTKLTVL | | |
| QSVLTQPPSASGTPGQRVTISCQGDSLRSYYVNW | VL of scFv | 27 |
| YQQLPGTAPKLLIYDHSKRPTGVPDRFSGSKSGT | (Light chain variable | |
| SASLAISGLRSEDEADYYCQSYDSSTVVFGGGTK | region 2) | |
| LTVLG | | |
| EIVLTQSPGTLSLSPGERATLSCKASQDIDDDIN | VL of scFv (light chain | 29 |
| WYQQKPGQAPRLLIYDASLRATGIPDRFSGSGSG | variable region 3) | |
| TDFTLTISRLEPEDFAVYYCQQSLRTPITFGQGT | | |
| KLEIKR | | |
| EIVLTQSPGTLSLSPGERATLSCRASQGIDSYVA | VL of scFv (light chain | 31 |
| WYQQKPGQAPRLLIYDASLRATGIPDRFSGSGSG | variable region 4) | |
| TDFTLTISRLEPEDFAVYYCQQYNSWPITFGQGT | | |
| KLEIKR | | |
| EVQLLESGGGLVQPGGSLRLSCAASGFTFSNYDM | VH of scFv (heavy chain | 26 |
| SWVRQAPGKGLEWVSWIYPSDSSIYYADSVKGRF | variable region 1) | |
| TISRDNSKNTLYLQMNSLRAEDTAVYYCARGPFA | | |
| NKYRQFDYWGQGTLVTVSS | | |
| EVQLLESGGGLVQPGGSLRLSCAASGFTFSNYGV | VH of scFv (heavy chain | 28 |
| HWVRQAPGKGLEWVSYISYSGGTYYNPSLKSRFT | variable region 2) | |
| ISRDNSKNTLYLQMNSLRAEDTAVYYCARDSDDF | | |
| GFDYWGQGTLVTVSS | | |
| EVQLLESGGGLVQPGGSLRLSCAASGFTFSDYGL | VH of scFv (heavy chain | 30 |
| SWVRQAPGKGLEWVSLIDSSGSSTFYADSVKGRF | variable region 3) | |
| TISRDNSKNTLYLQMNSLRAEDTAVYYCAKEHGL | | |
| FDSWGQGTLVTVSS | | |
| EVQLLESGGGLVQPGGSLRLSCAASGFTFSGHYW | VH of scFv (heavy chain | 32 |
| SWVRQAPGKGLEWVSTVSGSGGDTFYADSVKGRF | variable region 4) | |
| TISRDNSKNTLYLQMNSLRAEDTAVYYCARGHSV | | |
| MDVWGQGTLVTVSS | | |
| GSTSGSGKPSGEGSTKG | Linker peptide 1 | 37 |
| | Whitlow Linker | |
| GGGGS | Linker peptide 2 | 38 |
| EPKSCDKTHTCPPCP | IgG1 alpha hinge region | 39 |
| ESKYGPPCPSCP | IgG4 alpha hinge region | 40 |
| ALSNSIMYFSHFVPVFLPAKPTTTPAPRPPTPAP | CD8 alpha hinge region | 41 |
| TIASQPLSLRPEASRPAAGGAVHTRGLD | | |
| AWVSACDTEDTVGHLGPWRDKDPALWCQLCLSSQ | CD8 alpha transmembrane | 42 |
| HQAIERFYDKMQNAESGRGQVMSSLAELEDDFKE | domain | |
| GYLETVAAYYEE | | |
| KPFWVLVVVGGVLACYSLLVTVAFIIFWV | CD28 transmembrane domain | 43 |
| RSKRSRLLHSDYMNMTPRRPGPTRKHYQPYAPPR | CD28 co-stimulatory | 44 |
| DFAAYRS | domain | |
| GGGGS | Linker peptide 2 | 38 |
| LCARPRRSPAQEDGKVYINMPGRG | DAP10 co-stimulatory | 45 |
| | domain | |
| KRGRKKLLYIFKQPFMRPVQTTQEEDGCSCRFPE | CD137 co-stimulatory | 46 |
| EEEGGCEL | domain | |
| GGGGS | Linker peptide 2 | 38 |
| RVKFSRSADAPAYQQGQNQLYNELNLGRREEYDV | CD3 zeta signaling domain | 47 |
| LDKRRGRDPEMGGKPRRKNPQEGLYNELQKDKMA | | |
| EAYSEIGMKGERRRGKGHDGLYQGLSTATKDTYD | | |
| ALHMQALPPR | | |

Designs of 4 Types of BCMA-CARs

In particular, the domains of BCMA-CAR constructed and used in the experiment were designed as follows: (i) a CD16 signal peptide; (ii) a polypeptide including a heavy chain variable region (V_{H}) and a light chain variable region (V_{L}) of an scFv (single chain variant fragment) targeting BCMA, and a linker connecting the VH and the VL; (iii) a CD8α hinge region polypeptide; (iv) a CD28 transmembrane domain polypeptide; and (v) a polypeptide including an intracellular domain of CD28, a linker (GGGGS), an intracellular domain of CD137, a linker (GGGS), and an intracellular domain of CD3ζ. FIG. 1B schematically shows the structure of BCMA-CAR of the present invention.

Construction of BCMA-CAR1

BCMA-CAR1 was constructed with sequential connection of the following polypeptides (i) to (v).

(i) a CD16 signal peptide (SEQ ID NO: 33);

(ii) a heavy chain variable region (V_{H}) of a scFv targeting BCMA (SEQ ID NO: 26)—a linker peptide (GSTSGSGKPSGEGSTKG, SEQ ID NO: 37)—a light chain variable region (V_{L}) of a scFv targeting BCMA (SEQ ID NO: 25);

(iii) a CD8α hinge region polypeptide (SEQ ID NO: 41);

(iv) a CD28 transmembrane domain polypeptide (SEQ ID NO: 43); and

(v) an intracellular domain of CD28 (SEQ ID NO: 44)—a linker (GGGGS)—intracellular domain of CD137 (SEQ ID NO: 46)—a linker (GGGGS)—an intracellular domain of CD3ζ (SEQ ID NO: 47).

The amino acid sequences of the V_{L} and V_{H} of a scFV of BCMA-CAR1 are as follows.
Light chain variable region (V_{L}) amino acid sequence (110 aa):

(SEQ ID NO: 25)
QSVLTQPPSASGTPGQRVTISCSGSSSNIGSNSVSWYQQLPGTAPKLLI
YADSKRPSGVPDRFSGSKSGTSASLAISGLRSEDEADYYCGSWDYSLSG
YVFGGGTKLTVL
L-CDR1:
(SEQ ID NO: 1)
SGSSSNIGSNSVS
L-CDR2:
(SEQ ID NO: 2)
ADSKRPS
L-CDR3:
(SEQ ID NO: 3)
GSWDYSLSGYV

Heavy chain variable region (V_{H}) amino acid sequence (121 aa):

(SEQ ID NO: 26)
EVQLLESGGGLVQPGGSLRLSCAASGFTFSNYDMSWVRQAPGKGLEWVS
WIYPSDSSIYYADSVKGRFTISRDNSKNTLYLQMNSLRAEDTAVYYCAR
GPFANKYRQFDYWGQGTLVTVSS
H-CDR1:
(SEQ ID NO: 4)
NYDMS
H-CDR2:
(SEQ ID NO: 5)
WIYPSDSSIYYADSVKG
H-CDR3:
(SEQ ID NO: 6)
RGPFANKYRQFDY

The entire amino acid sequence of BCMA-CAR1 is set forth in SEQ ID NO: 48.

Construction of BCMA-CAR2

BCMA-CAR2 was constructed with the following polypeptides (i) to (v) sequentially connected.

(i) a CD16 signal peptide (SEQ ID NO: 33);

(ii) a heavy chain variable region (V_{H}) of a scFv targeting BCMA (SEQ ID NO: 28)—a linker peptide (GSTSGSGKPSGEGSTKG, SEQ ID NO: 37)—a light chain variable region (V_{L}) of a scFv targeting BCMA (SEQ ID NO: 27);

(iii) a CD8α hinge region polypeptide (SEQ ID NO: 41);

(iv) a CD28 transmembrane domain polypeptide (SEQ ID NO: 43); and

(v) an intracellular domain of CD28 (SEQ ID NO: 44)—a linker (GGGGS)—an intracellular domain of CD137 (SEQ ID NO: 46)—a linker (GGGGS)—an intracellular domain of CD3ζ (SEQ ID NO: 47).

The amino acid sequences of a V_{L} and V_{H} of a scFV of BCMA-CAR2 are as follows.
Light chain variable region (V_{L}) amino acid sequence (107 aa):

(SEQ ID NO: 27)
QSVLTQPPSASGTPGQRVTISCQGDSLRSYYVNWYQQLPGTAPKLLIYD
HSKRPTGVPDRFSGSKSGTSASLAISGLRSEDEADYYCQSYDSSTVVFG
GGTKLTVLG
L-CDR1:
(SEQ ID NO: 7)
QGDSLRSYYVN
L-CDR2:
(SEQ ID NO: 8)
DHSKRPT
L-CDR3:
(SEQ ID NO: 9)
QSYDSSTV

Heavy chain variable region (V_{H}) amino acid sequence (117 aa):

(SEQ ID NO: 28)
EVQLLESGGGLVQPGGSLRLSCAASGFTFSNYGVHWVRQAPGKGLEWVS
YISYSGGTYYNPSLKSRFTISRDNSKNTLYLQMNSLRAEDTAVYYCARD
SDDFGFDYWGQGTLVTVSS
H-CDR1:
(SEQ ID NO: 10)
NYGVH
H-CDR2:
(SEQ ID NO: 11)
YISYSGGTYYNPSLKS
H-CDR3:
(SEQ ID NO: 12)
RDSDDFGFDY

The entire amino acid sequence of BCMA-CAR2 is set forth in SEQ ID NO: 49.

Construction of BCMA-CAR3

BCMA-CAR3 was constructed with sequential connection of the following polypeptides (i) to (v).

(i) a CD16 signal peptide (SEQ ID NO: 33);

(ii) a heavy chain variable region (V_{H}) of a scFv targeting BCMA (SEQ ID NO: 30)—a linker peptide (GSTSGSGKPSGEGSTKG, SEQ ID NO: 37)—a light chain variable region (V_{L}) of a scFv targeting BCMA (SEQ ID NO: 29);

(iii) a CD8α hinge region polypeptide (SEQ ID NO: 41);

(iv) a CD28 transmembrane domain polypeptide (SEQ ID NO: 43); and

(v) an intracellular domain of CD28 (SEQ ID NO: 44)—a linker (GGGGS)—intracellular domain of CD137 (SEQ ID NO: 46)—a linker (GGGGS)—an intracellular domain of CD3ζ (SEQ ID NO: 47).

The amino acid sequences of a VL and VH of a scFV of BCMA-CAR3 are as follows.
Light chain variable region (V_{L}) amino acid sequence (108 aa):

(SEQ ID NO: 29)
EIVLTQSPGTLSLSPGERATLSCKASQDIDDDINWYQQKPGQAPRLLIY
DASLRATGIPDRFSGSGSGTDFTLTISRLEPEDFAVYYCQQSLRTPITF
GQGTKLEIKR
L-CDR1:
(SEQ ID NO: 13)
KASQDIDDDIN
L-CDR2:
(SEQ ID NO: 14)
DASLRAT
L-CDR3:
(SEQ ID NO: 15)
QQSLRTPI

Heavy chain variable region (V_{H}) amino acid sequence (121 aa):

(SEQ ID NO: 30)
EVQLLESGGGLVQPGGSLRLSCAASGFTFSDYGLSWVRQAPGKGLEWVS
LIDSSGSSTFYADSVKGRFTISRDNSKNTLYLQMNSLRAEDTAVYYCAK
EHGLFDSWGQGTLVTVSS
H-CDR1:
(SEQ ID NO: 16)
DYGLS
H-CDR2:
(SEQ ID NO: 17)
LIDSSGSSTFYADSVKG
H-CDR3:
(SEQ ID NO: 18)
KEHGLFDS

The entire amino acid sequence of BCMA-CAR3 is set forth in SEQ ID NO: 50.

Construction of BCMA-CAR4

BCMA-CAR4 was constructed with the following polypeptides (i) to (v) sequentially connected.

(i) a CD16 signal peptide (SEQ ID NO: 33);

(ii) a heavy chain variable region (V_{H}) of a scFv targeting BCMA (SEQ ID NO: 32)—a linker peptide (GSTSGSGKPSGEGSTKG, SEQ ID NO: 37)—a light chain variable region (V_{L}) of a scFv targeting BCMA (SEQ ID NO: 31);

(iii) a CD8α hinge region polypeptide (SEQ ID NO: 41);

(iv) a CD28 transmembrane domain polypeptide (SEQ ID NO: 43); and

(v) an intracellular domain of CD28 (SEQ ID NO: 44)—a linker (GGGGS)—intracellular domain of CD137 (SEQ ID NO: 46)—a linker (GGGGS)—an intracellular domain of CD3ζ (SEQ ID NO: 47).

The amino acid sequences of a VL and VH of a scFV of BCMA-CAR4 are as follows.
Light chain variable region (V_{L}) amino acid sequence (108 aa):

(SEQ ID NO: 31)
EIVLTQSPGTLSLSPGERATLSCRASQGIDSYVAWYQQKPGQAPRLLIY
DASLRATGIPDRFSGSGSGTDFTLTISRLEPEDFAVYYCQQYNSWPITF
GQGTKLEIKR
L-CDR1:
(SEQ ID NO: 19)
RASQGIDSYVA
L-CDR2:
(SEQ ID NO: 20)
DASLRAT
L-CDR3:
(SEQ ID NO: 21)
QQYNSWPI

Heavy chain variable region (V_{H}) amino acid sequence (116 aa):

(SEQ ID NO: 32)
EVQLLESGGGLVQPGGSLRLSCAASGFTFSGHYWSWVRQAPGKGLEWVS
TVSGSGGDTFYADSVKGRFTISRDNSKNTLYLQMNSLRAEDTAVYYCAR
GHSVMDVWGQGTLVTVSS
H-CDR1:
(SEQ ID NO: 22)
GHYWS
H-CDR2:
(SEQ ID NO: 23)
TVSGSGGDTFYADSVKG
H-CDR3:
(SEQ ID NO: 24)
RGHSVMDV

The entire amino acid sequence of BCMA-CAR4 is set forth in SEQ ID NO: 51.

The polypeptide sequence of BCMA-CAR with the above configuration was codon-optimized to be suitable for protein expression in animal cells and thus converted to amino acid sequences, and then cloned into pSBbi-Neo (Addgene, Catalog No. 60525), which is a transposon vector containing an EF1a promoter.

### Example 2: Construction of BCMA-CAR NK Cells

Construction of 4 types of BCMA-CAR expressing NK cells

Nucleic acid sequences (gene) encoding the 4 types of anti-BCMA CARs prepared in Example 1, BCMA-CAR1, BCMA-CAR2, BCMA-CAR3, and BCMA-CAR4, were cloned into pSBbi-Noe vectors and were subjected to electroporation using Nucleofector and Cell line Nucleofector Kit manufactured by Lonza, together with sleeping beauty transposase-expression vector pCMV(CAT)T7-SB100 (Addgene, Catalog No. 34879), to prepare 4 types of BCMA-CAR NK cells, CGT301, CGT302, CGT303, and CGT304, respectively.

Meanwhile, the reference BCMA-CAR NK cell (Ref) for evaluating the efficacy of the present BCMA-CAR NK cells was prepared by using a BCMA-CAR having the same structure as the BCMA-CAR of the present invention except for scFv which is prepared based on J6Mo antibody sequence of BCMA-ADC (GSK2857916, BLENREP).

After transfection, cells were stabilized for 48 hours in α-MEM media containing 12.5% FBS, 12.5% horse serum, 0.1 mM 2-mercaptoethanol, 1% penicillin/streptomycin, and IL-2 100 IU/ml, and only NK-92 cells expressing BCMA-CAR gene were selected by treatment with Kanamycin at a concentration of 200 μg/ml for 2 weeks or more.

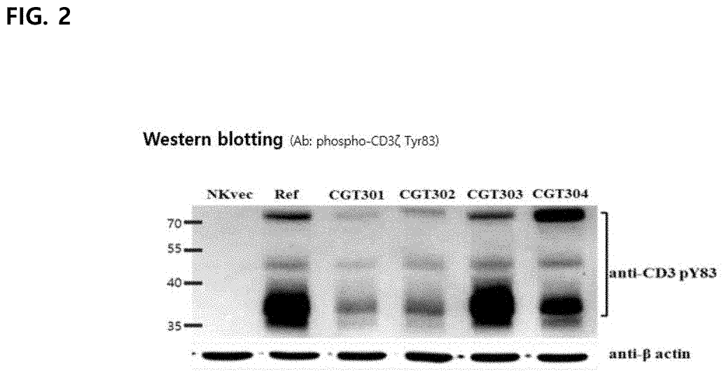

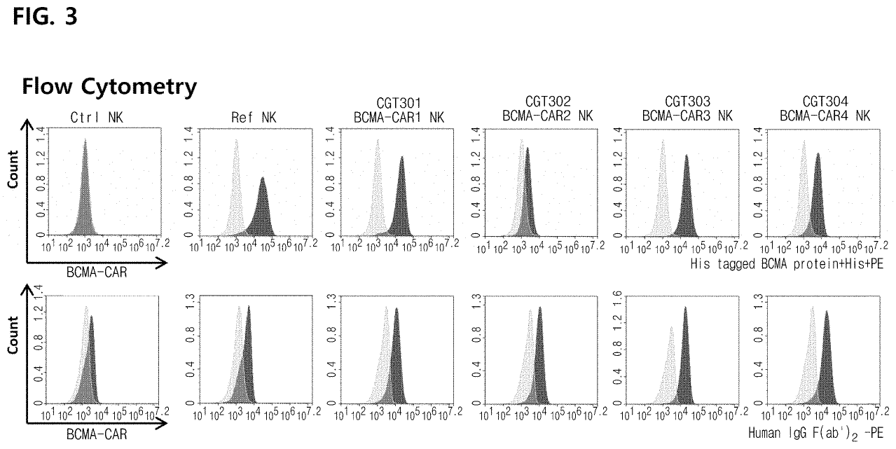

The prepared BCMA-CAR NK cells were first subjected to Western blotting using anti-phospho-CD3ζ antibodies to measure BCMA-CAR expression in the NK cells (FIG. 2). Also, BCMA-CAR expression on the NK cell surface was measured by flow cytometry using human IgG(ab′)2 antibody, or His tag conjugated-BCMA recombinant protein and PE-conjugated His antibodies (FIG. 3).

### Example 3: Evaluation of In Vitro Efficacy of NK Cells Expressing BCMA-CAR

Cytolytic Activity of BCMA-CAR NK Toward Multiple Myeloma Cells

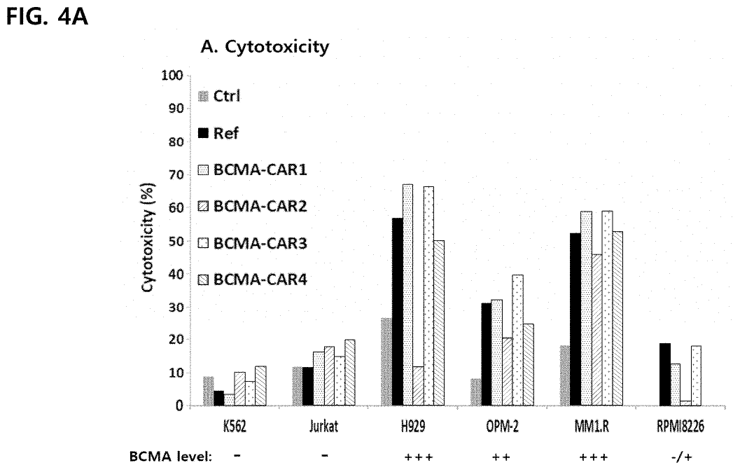

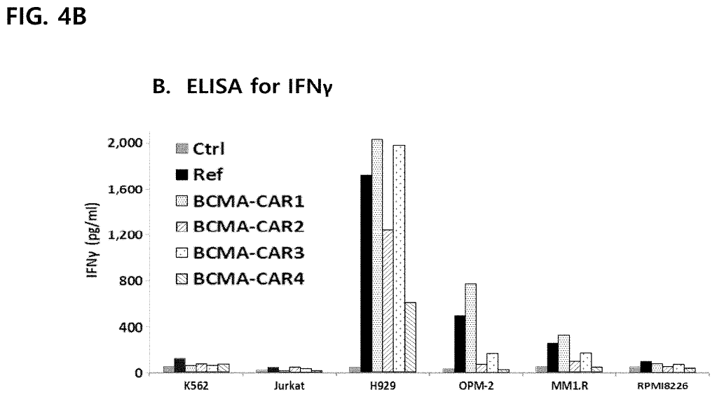

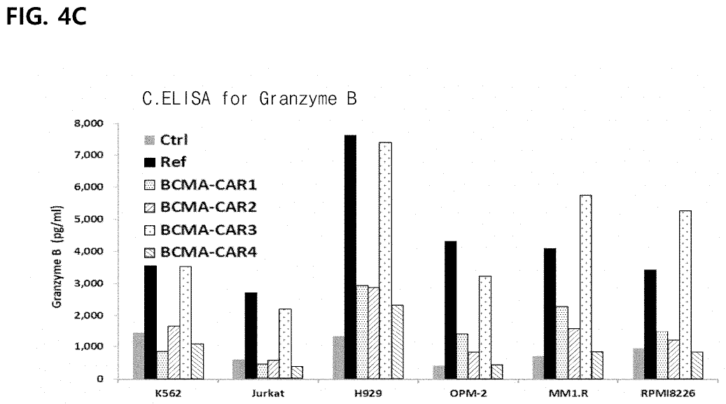

To evaluate selectivity and reactivity of the 4 types of BCMA-CAR NK cells prepared in Example 2 in response to multiple myeloma, the BCMA-CAR NK cells were reacted with multiple myeloma cells, H929, MM1.R, and OPM-2, as well as with other cancer cells, K562 and Jurkat, and then were subjected to a cytotoxicity assay and measured for the amount of Granzyme B and IFNγ released. The result showed that while the BCMA-CAR NK cells showed no difference in reactivity compared to that of control group (Ctrl) NK cells (NKvec) having vector without BCMA-CAR as to the cancer cell lines lacking BCMA expression, e.g., K562 and Jurkat, the BCMA-CAR NK cells showed cytolytic activity 3-4 times or higher than that of control group (Ctrl) NK cells (NKvec) as to the multiple myeloma cell lines having high BCMA expression levels, e.g., H929, MM1.R, and OPM-2 (FIG. 4A). It could be confirmed that the cytolytic activity of BCMA-CAR NK specific to multiple myeloma tumor cells is attributed to increased amounts of Granzyme B and IFNγ released (FIG. 4B, FIG. 4C).

Preparation of K562-BCNA Target Cell Lines

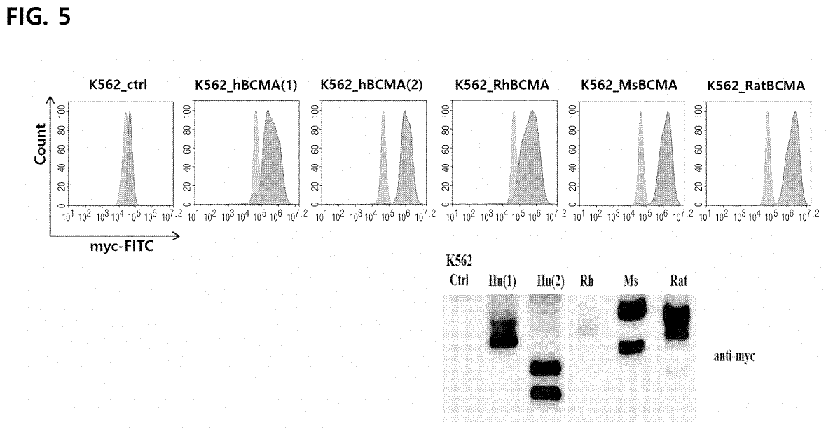

To evaluate BCMA antigen-specific efficacy of the BCMA-CAR designed in Example 1, first, target cell lines expressing BCMA antigens derived from different species were prepared. In K562 cancer cells lacking BCMA expression, the BCMA gene expressing myc was introduced at the end of BCMA derived from human (h), monkey (Rh), or mouse (Ms) and rat (Rat), respectively, and the expression thereof was measured by flow cytometry and Western blotting (FIG. 5).

Analysis of BCA Antigen Specificity of 4 Types of BCA-CARs

To analyze antigen-specificity of the 4 types of BCMA-CARs prepared in Example 1, BCMA-CAR1 NK cells, BCMA-CAR2 NK cells, BCMA-CAR3 NK cells, or BCMA-CAR4 NK cells were reacted with K562 parent cells or K562 (K562-BCMA) cell line expressing BCMA derived from human (hu1, hu2), monkey (Rh), mouse (Ms), and rat (Rat), and then a cytotoxicity assay was performed, and the amount of Granzyme B and IFNγ released was assayed.

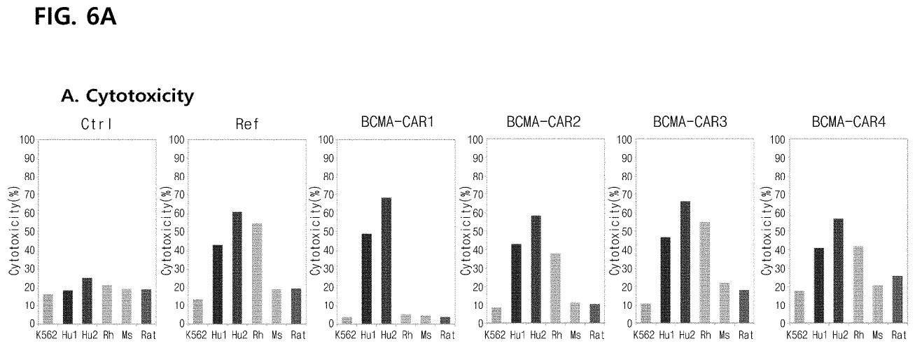

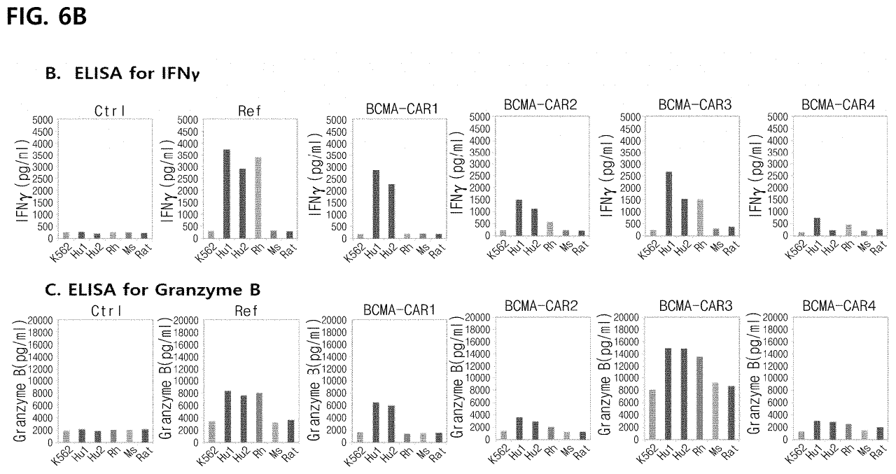

BCMA-CAR1 NK cells expressing BCMA-CAR1 showed activity specifically only in the human-derived BCMA, and the NK cells expressing BCMA-CAR2, BCMA-CAR3, and BCMA-CAR4, respectively, showed reactivity to non-human BCMA antigens (FIG. 6A and FIG. 6B). Meanwhile, the reference BCMA-CAR NK cell (Ref) having scFv derived from BCMA antigen (J6MO) manufactured by GSK showed reactivity similar to those of BCMA-CAR2, BCMA-CAR3, and BCMA-CAR4.

### Example 4: Verification of BCNA-Specific Cytolytic Activity of BCNA-CAR NK Cells

Confirmation of BCNA-Specific Activity of BCNA-CAR NK Using BCNA Recombinant Protein

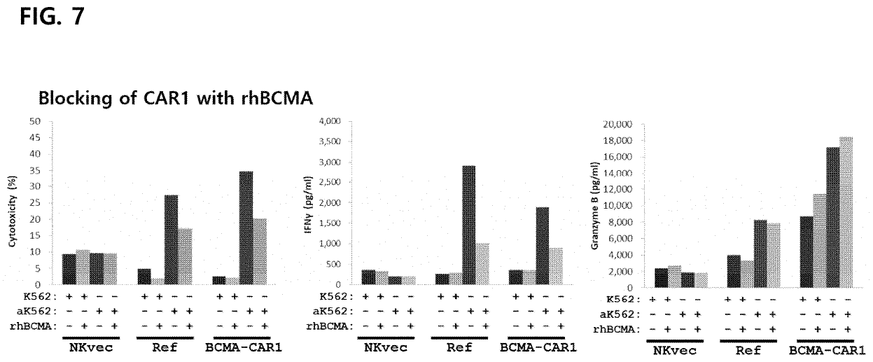

To determine whether an activity displayed by BCMA-CAR1 NK cells is BCMA-specific, a competition assay using rhBCMA was performed. First, BCMA-CAR NK cells (4×10⁴ cells) were pre-incubated for 1 hour with 1 μg of rhBCMA protein added, and after addition of K562-BCMA cells (aK562, 4×10⁴ cells) overexpressing human BCMA as target cells, were allowed to react for 4 hours and then measured for cytotoxicity. The result showed that in the presence of rhBCMA proteins, tumor cytolytic activity of BCMA-CAR NK cells was reduced by about 40% (FIG. 7).

Confirmation of BCMA-Specific Activity of BCM-CAR NK Using BCMA Antibodies

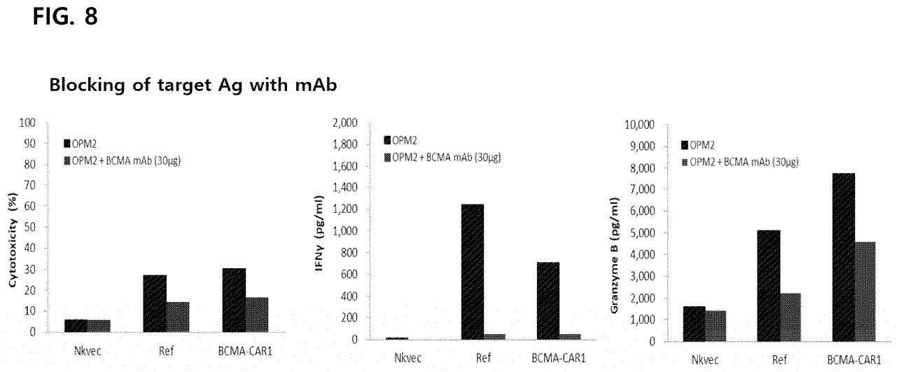

In particular, using OPM-2, multiple myeloma cells with a high BCMA expression as target cancer cells, OPM-2 cells (4×10⁴ cells) were pre-incubated for 1 hour with 30 μg of anti-BCMA mAb (J6M0) added, and then after addition of BCMA-CAR1 NK cells (4×10⁴ cells), were allowed to react for 4 hours and then measured for cytotoxicity. The result showed that in the presence of BCMA antibody (anti-BCMA mAb), the cytotoxicity of BCMA-CAR NK cells toward OPM-2 multiple myeloma cells was reduced by about 50%, and similarly, the amount of Granzyme B released was reduced by about 50%, and in particular, the amount of IFNγ released was reduced to 1/20 (FIG. 8).

These experiment results are attributed to the BCMA-specific anticancer activity of BCMA-CAR NK cells by binding of BCMA-CAR1 receptors expressed on the NK cell surface to the BCMAs which are antigens specifically expressed on the surface of multiple myeloma cancer cells.

### Example 5: Evaluation of In Vivo Anticancer Efficacy of BCMA-CAR NK Cells

In a xenograft animal model using NOD.Cg-Prkdcscid Il2rgtm1Wj1/SzJ (NSG) mouse (Jackson Laboratory, Jax #00557 strain), the anticancer efficacy of BCMA-CAR NK cells expressing BCMA-CAR1 was evaluated.

After subcutaneous implantation of multiple myeloma cell line H929 with high BCMA expression, when the tumor size reached 150 mm³ (10 days post implantation), 5 mice were randomly assigned to each group, and once every 5 days, BCMA-CAR NK cells or control NK cells (NKvec) were administered at a concentration of 1×10⁷ cells via intravenous tail vein injection (i.v.) in a total of 3 administrations. Here, a proteasome inhibitor, Velcade® (ingredient name: Bortezomib) and an antibody medication, Darzalex® (ingredient name: Daratumumab) being used as treatment medications for multiple myeloma patients were used as control drugs, and Velcade® (Vel) and Darzalex® (Dara) were each administered at a concentration of 0.5 mg/kg via intraperitoneal (i.p.) route, once every 3-4 days, in a total of 5 administrations.

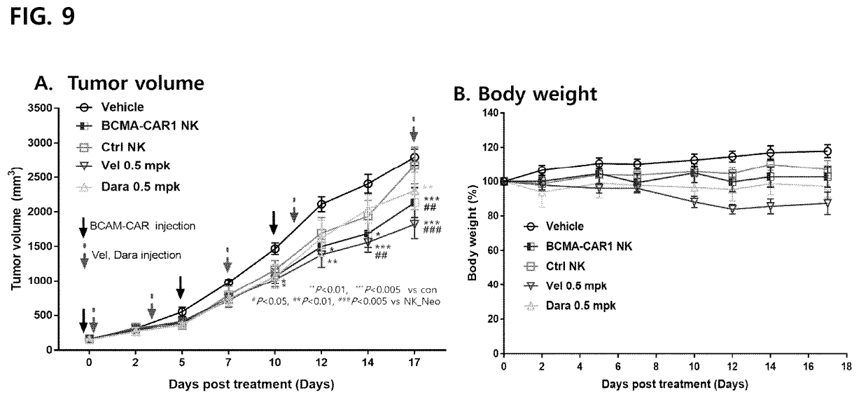

The result of comparison of tumor sizes at day 17 in the experiment showed while the test group (BCMA-CAR1 NK) administered with BCMA-CAR NK cells showed a 20-times increase compared to a pre-treatment tumor size, the untreated group (Vehicle) showed a 26-times increase and the group treated with NK cells (NKctrl) lacking a therapeutic gene expression showed a 25-times increase. On the last day of experiment (Day 17), comparison of the growth inhibition rate (GIR (%)) of each treatment group versus untreated group (Vehicle) showed that, while the experiment group administered with BCMA-CAR NK cells (BCMA-CAR1 NK) showed 25% of tumor growth inhibition, the group administered with control NK cells (NKctrl) showed 6%, the group administered with Velcade® (Vel) showed 39%, and the group administered with Darzalex® (Dara) showed 23% of tumor growth inhibition (FIG. 9). Velcade, which is used as primary therapy for MM (multiple myeloma), showed excellent anticancer efficacy of 39% but showed toxicity i.e., a decrease in body weight, while BCMA-CAR NK cells showed a comparable level of anticancer efficacy as the antibody medication Darzalex without toxicity.

### Example 6: Analysis of Characteristics of BCNA-CAR NK Cells

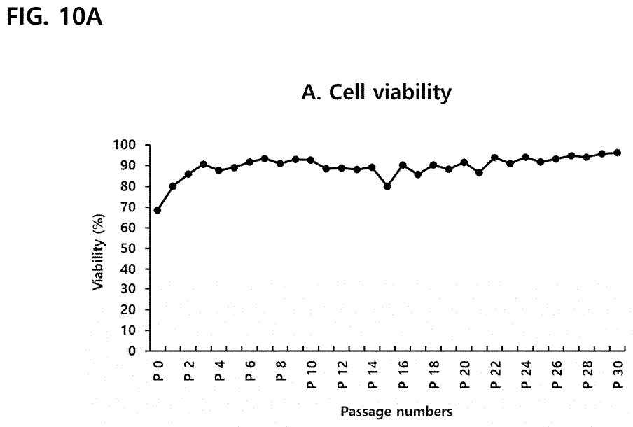

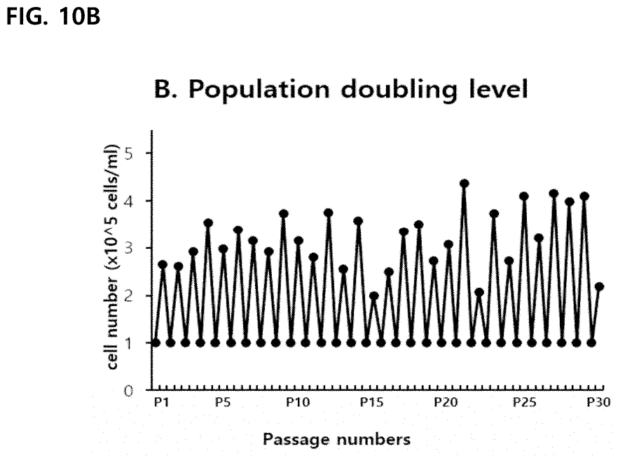

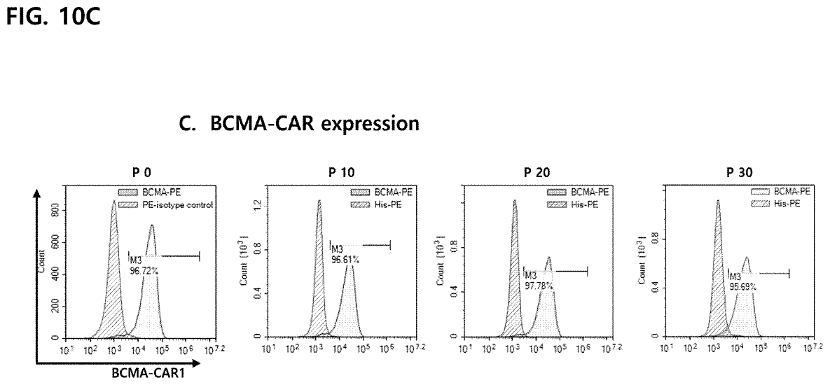

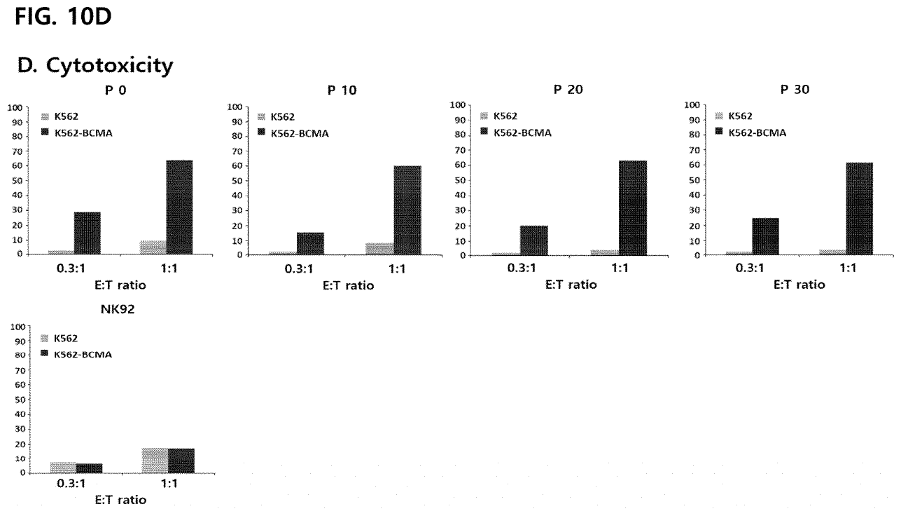

To analyze characteristics of BCMA-CAR NK cells, the BCMA-CAR NK cells were cultured for 60 days continuously and were evaluated for cell viability, proliferation, CAR expression, cell activity (cytotoxicity), and the like. The result showed that even after 60 days, the BCMA-CAR NK cells maintained 95% or more of CAR expression and were tripled in cell count every 2 days, and also maintained cytotoxicity, which kills cancer cells expressing BCMA antigens, at a constant level of 60% or more. It was confirmed that the BCMA-CAR NK cells of the present invention have characteristics that not only cell proliferation, but also CAR expression and cell activity remain stable for 60 days or longer (FIG. 10A to FIG. 10D).

1. A chimeric antigen receptor (CAR), comprising the following polypeptides:
(i) an extracellular antigen binding domain that specifically binds to a B cell maturation antigen (BCMA);
(ii) a transmembrane domain;
(iii) an intracellular co-stimulatory signaling domain; and
(iv) an intracellular primary signaling domain.

2. The CAR of claim 1, wherein the extracellular antigen binding domain comprises an anti-BCMA antibody or antigen binding fragment thereof;
wherein the extracellular antigen binding domain comprises:
(i) a light chain variable region (V_{L}) of an anti-BCMA antibody, comprising L-CDR (complementarity determining region) 1, L-CDR2, and L-CDR3;
(ii) a heavy chain variable region (V_{H}) of the anti-BCMA antibody, including H-CDR1, H-CDR2, and H-CDR3; or
(iii) at least one of the V_{L} and at least one of the V_{H}.

3. (canceled)

4. The CAR of claim 2, wherein
the L-CDR1 is a polypeptide having the amino acid sequence of SEQ ID NO: 1, SEQ ID NO: 7, SEQ ID NO: 13, or SEQ ID NO: 19;
the L-CDR2 is a polypeptide having the amino acid sequence of SEQ ID NO: 2, SEQ ID NO: 8, SEQ ID NO: 14, or SEQ ID NO: 20;
the L-CDR3 is a polypeptide having the amino acid sequence of SEQ ID NO: 3, SEQ ID NO: 9, SEQ ID NO: 15, or SEQ ID NO: 21;
the H-CDR1 is a polypeptide having the amino acid sequence of SEQ ID NO: 4, SEQ ID NO: 10, SEQ ID NO: 16, or SEQ ID NO: 22;
the H-CDR2 is a polypeptide having the amino acid sequence of SEQ ID NO: 5, SEQ ID NO: 11, SEQ ID NO: 17, or SEQ ID NO: 23; and
the H-CDR3 is a polypeptide having the amino acid sequence of SEQ ID NO: 6, SEQ ID NO: 12, SEQ ID NO: 18, or SEQ ID NO: 24.

5. The CAR of claim 2, wherein the extracellular antigen binding domain comprises:
a heavy chain variable region (V_{H}) of the anti-BCMA antibody, including H-CDR1 having the amino acid sequence of SEQ ID NO: 4, H-CDR2 having the amino acid sequence of SEQ ID NO: 5, and H-CDR3 having the amino acid sequence of SEQ ID NO: 6, and a light chain variable region (V_{L}) of the anti-BCMA antibody, including L-CDR1 having the amino acid sequence of SEQ ID NO: 1, L-CDR2 having the amino acid sequence of SEQ ID NO: 2, and L-CDR3 having the amino acid sequence of SEQ ID NO: 3;
a heavy chain variable region (V_{H}) of the anti-BCMA antibody, including H-CDR1 having the amino acid sequence of SEQ ID NO: 10, H-CDR2 having the amino acid sequence of SEQ ID NO: 11, and H-CDR3 having the amino acid sequence of SEQ ID NO: 12, and a light chain variable region (V_{L}) of the anti-BCMA antibody, including L-CDR1 having the amino acid sequence of SEQ ID NO: 7, L-CDR2 having the amino acid sequence of SEQ ID NO: 8, and L-CDR3 having the amino acid sequence of SEQ ID NO: 9;
a heavy chain variable region (V_{H}) of the anti-BCMA antibody, including H-CDR1 having the amino acid sequence of SEQ ID NO: 16, H-CDR2 having the amino acid sequence of SEQ ID NO: 17, and H-CDR3 having the amino acid sequence of SEQ ID NO: 18, and a light chain variable region (V_{L}) of the anti-BCMA antibody, including L-CDR1 having the amino acid sequence of SEQ ID NO: 13, L-CDR2 having the amino acid sequence of SEQ ID NO: 14, and L-CDR3 having the amino acid sequence of SEQ ID NO: 15; and
a heavy chain variable region (V_{H}) of the anti-BCMA antibody, including H-CDR1 having the amino acid sequence of SEQ ID NO: 22, H-CDR2 having the amino acid sequence of SEQ ID NO: 23, and H-CDR3 having the amino acid sequence of SEQ ID NO: 24, and a light chain variable region (V_{L}) of the anti-BCMA antibody, including L-CDR1 having the amino acid sequence of SEQ ID NO: 19, L-CDR2 having the amino acid sequence of SEQ ID NO: 20, and L-CDR3 having the amino acid sequence of SEQ ID NO: 21.

6. The CAR of claim 1, wherein the extracellular antigen binding domain comprises one, or two or more of:
(i) at least one light chain variable region (V_{L}) of the anti-BCMA antibody, selected from the group consisting of polypeptides having the amino acid sequences of SEQ ID NO: 25, SEQ ID NO: 27, SEQ ID NO: 29, and SEQ ID NO: 31; and
(ii) at least one heavy chain variable region (V_{H}) of the anti-BCMA antibody, selected from the group consisting of polypeptides having the amino acid sequences of SEQ ID NO: 26, SEQ ID NO: 28, SEQ ID NO: 30, and SEQ ID NO: 32.

7. The CAR of claim 1, further comprising a linker located between two or more light chain or heavy chain variable regions; wherein the linker is a polypeptide having the amino acid sequence of SEQ ID NO: 37 or SEQ ID NO: 38.

8. (canceled)

9. The CAR of claim 1, wherein the extracellular antigen binding domain further comprises a hinge region connected to the transmembrane domain; wherein the hinge region comprises an IgG1 hinge region, an IgG4 hinge region, or a CD8α hinge region.

10. (canceled)

11. The CAR of claim 9,
wherein the IgG1 hinge region is a polypeptide having the amino acid sequence of SEQ ID NO: 39;
the IgG4 hinge region is a polypeptide having the amino acid sequence of SEQ ID NO: 40; and
the CD8α hinge region is a polypeptide having the amino acid sequence of SEQ ID NO: 41.

12. The CAR of claim 1, wherein the transmembrane domain comprises a CD8α transmembrane region or a CD28 transmembrane domain region.

13. The CAR of claim 12, wherein the CD8α transmembrane region is a polypeptide having the amino acid sequence of SEQ ID NO: 42, and the CD28 transmembrane region is a polypeptide having the amino acid sequence of SEQ ID NO: 43.

14. The CAR of claim 1, wherein the intracellular co-stimulatory signaling domain comprises an intracellular co-stimulatory signaling domain of CD28, an intracellular co-stimulatory signaling domain of DAP10, or an intracellular co-stimulatory signaling domain of CD137(4-1BB).

15. The CAR of claim 14,
wherein the intracellular co-stimulatory signaling domain of CD28 is a polypeptide having the amino acid sequence of SEQ ID NO: 44;
the intracellular co-stimulatory signaling domain of DAP10 is a polypeptide having the amino acid sequence of SEQ ID NO: 45; and
the intracellular co-stimulatory signaling domain of CD137 (4-1BB) is a polypeptide having the amino acid sequence of SEQ ID NO: 46.

16. The CAR of claim 1, wherein the intracellular primary signaling domain comprises an intracellular domain of CD3ζ; wherein the intracellular signaling domain of CD3ζ is a polypeptide having the amino acid sequence of SEQ ID NO: 47.

17. (canceled)

18. The CAR of claim 1, wherein the intracellular co-stimulatory signaling domain and the intracellular primary signaling domain are connected via a linker; wherein the linker is a polypeptide having the amino acid sequence of SEQ ID NO: 38.

19. (canceled)

20. The CAR of claim 1, wherein the extracellular antigen binding domain further comprises a signal peptide:
wherein the signal peptide comprises a CD16 signal peptide, a GM-CSF signal peptide, a human IgG signal peptide, or a CD8 signal peptide;
wherein the CD16 signal peptide is a polypeptide having the amino acid sequence of SEQ ID NO: 33; the GM-CSF signal peptide is a polypeptide having the amino acid sequence of SEQ ID NO: 34; the human IgG signal peptide is a polypeptide having the amino acid sequence of SEQ ID NO: 35; and the CD8α signal peptide is a polypeptide having the amino acid sequence of SEQ ID NO: 36.

21. (canceled)

22. (canceled)

23. A nucleic acid molecule encoding the CAR of claim 1.

24. A vector comprising the nucleic acid molecule of claim 23.

25. A cell comprising the nucleic acid molecule of claim 23; wherein the cell is an immune cell, and the immune cell is an NK (natural killer) cell or a T cell.

26. (canceled)

27. (canceled)

28. A pharmaceutical composition for treating or preventing a B cell-related condition, comprising: (i) a pharmaceutically effective amount of the cell of claim 25; and (ii) a pharmaceutically acceptable carrier.

29. The pharmaceutical composition of claim 28, wherein the B cell-related condition is multiple myeloma, non-Hodgkin's lymphoma, B cell proliferation of uncertain malignant potential, lymphomatoid granulomatosis, posttransplant lymphoproliferative disorder, immunoregulatory disorders, rheumatoid arthritis, myasthenia gravis, idiopathic thrombocytopenic purpura, antiphospholipid syndrome, Chagas' disease, Grave's disease, Wegener's granulomatosis, polyarteritis nodosa, Sjogren's syndrome, pemphigus vulgaris, scleroderma, multiple sclerosis, antiphospholipid syndrome, ANCA-associated vasculitis, Goodpasture's disease, Kawasaki disease, autoimmune hemolytic anemia, and rapidly progressive glomerulonephritis, heavy chain disease, primary or immune cell-associated amyloidosis, or monoclonal gammopathy of undetermined significance.

30. (canceled)

31. (canceled)


SEQUENCE LISTING (51 sequences)

SEQ ID NO: 1 - protein, 13 aa, Artificial Sequence
  L-CDR1
SGSSSNIGSNSVS

SEQ ID NO: 2 - protein, 7 aa, Artificial Sequence
  L-CDR2
ADSKRPS

SEQ ID NO: 3 - protein, 11 aa, Artificial Sequence
  L-CDR3
GSWDYSLSGYV

SEQ ID NO: 4 - protein, 5 aa, Artificial Sequence
  H-CDR1
NYDMS

SEQ ID NO: 5 - protein, 17 aa, Artificial Sequence
  H-CDR2
WIYPSDSSIYYADSVKG

SEQ ID NO: 6 - protein, 13 aa, Artificial Sequence
  H-CDR3
RGPFANKYRQFDY

SEQ ID NO: 7 - protein, 11 aa, Artificial Sequence
  L-CDR1
QGDSLRSYYVN

SEQ ID NO: 8 - protein, 7 aa, Artificial Sequence
  L-CDR2
DHSKRPT

SEQ ID NO: 9 - protein, 8 aa, Artificial Sequence
  L-CDR3
QSYDSSTV

SEQ ID NO: 10 - protein, 5 aa, Artificial Sequence
  H-CDR1
NYGVH

SEQ ID NO: 11 - protein, 16 aa, Artificial Sequence
  H-CDR2
YISYSGGTYYNPSLKS

SEQ ID NO: 12 - protein, 10 aa, Artificial Sequence
  H-CDR3
RDSDDFGFDY

SEQ ID NO: 13 - protein, 11 aa, Artificial Sequence
  L-CDR1
KASQDIDDDIN

SEQ ID NO: 14 - protein, 7 aa, Artificial Sequence
  L-CDR2
DASLRAT

SEQ ID NO: 15 - protein, 8 aa, Artificial Sequence
  L-CDR3
QQSLRTPI

SEQ ID NO: 16 - protein, 5 aa, Artificial Sequence
  H-CDR1
DYGLS

SEQ ID NO: 17 - protein, 17 aa, Artificial Sequence
  H-CDR2
LIDSSGSSTFYADSVKG

SEQ ID NO: 18 - protein, 8 aa, Artificial Sequence
  H-CDR3
KEHGLFDS

SEQ ID NO: 19 - protein, 11 aa, Artificial Sequence
  L-CDR1
RASQGIDSYVA

SEQ ID NO: 20 - protein, 7 aa, Artificial Sequence
  L-CDR2
DASLRAT

SEQ ID NO: 21 - protein, 8 aa, Artificial Sequence
  L-CDR3
QQYNSWPI

SEQ ID NO: 22 - protein, 5 aa, Artificial Sequence
  H-CDR1
GHYWS

SEQ ID NO: 23 - protein, 17 aa, Artificial Sequence
  H-CDR2
TVSGSGGDTFYADSVKG

SEQ ID NO: 24 - protein, 8 aa, Artificial Sequence
  H-CDR3
RGHSVMDV

SEQ ID NO: 25 - protein, 110 aa, Artificial Sequence
  VL 1
QSVLTQPPSASGTPGQRVTISCSGSSSNIGSNSVSWYQQLPGTAPKLLIYADSKRPSGVPDRFSGSKSGTSASLAISGLRSEDEADYYCGSWDYSLSGYVFGGGTKLTVL

SEQ ID NO: 26 - protein, 121 aa, Artificial Sequence
  VH 1
EVQLLESGGGLVQPGGSLRLSCAASGFTFSNYDMSWVRQAPGKGLEWVSWIYPSDSSIYYADSVKGRFTISRDNSKNTLYLQMNSLRAEDTAVYYCARGPFANKYRQFDYWGQGTLVTVSS

SEQ ID NO: 27 - protein, 107 aa, Artificial Sequence
  VL 2
QSVLTQPPSASGTPGQRVTISCQGDSLRSYYVNWYQQLPGTAPKLLIYDHSKRPTGVPDRFSGSKSGTSASLAISGLRSEDEADYYCQSYDSSTVVFGGGTKLTVLG

SEQ ID NO: 28 - protein, 117 aa, Artificial Sequence
  VH 2
EVQLLESGGGLVQPGGSLRLSCAASGFTFSNYGVHWVRQAPGKGLEWVSYISYSGGTYYNPSLKSRFTISRDNSKNTLYLQMNSLRAEDTAVYYCARDSDDFGFDYWGQGTLVTVSS

SEQ ID NO: 29 - protein, 108 aa, Artificial Sequence
  VL 3
EIVLTQSPGTLSLSPGERATLSCKASQDIDDDINWYQQKPGQAPRLLIYDASLRATGIPDRFSGSGSGTDFTLTISRLEPEDFAVYYCQQSLRTPITFGQGTKLEIKR

SEQ ID NO: 30 - protein, 116 aa, Artificial Sequence
  VH 3
EVQLLESGGGLVQPGGSLRLSCAASGFTFSDYGLSWVRQAPGKGLEWVSLIDSSGSSTFYADSVKGRFTISRDNSKNTLYLQMNSLRAEDTAVYYCAKEHGLFDSWGQGTLVTVSS

SEQ ID NO: 31 - protein, 108 aa, Artificial Sequence
  VL 4
EIVLTQSPGTLSLSPGERATLSCRASQGIDSYVAWYQQKPGQAPRLLIYDASLRATGIPDRFSGSGSGTDFTLTISRLEPEDFAVYYCQQYNSWPITFGQGTKLEIKR

SEQ ID NO: 32 - protein, 116 aa, Artificial Sequence
  VH 4
EVQLLESGGGLVQPGGSLRLSCAASGFTFSGHYWSWVRQAPGKGLEWVSTVSGSGGDTFYADSVKGRFTISRDNSKNTLYLQMNSLRAEDTAVYYCARGHSVMDVWGQGTLVTVSS

SEQ ID NO: 33 - protein, 16 aa, Artificial Sequence
  CD16 signal peptide
MWQLLLPTALLLLVSA

SEQ ID NO: 34 - protein, 17 aa, Artificial Sequence
  GM-CSF signal peptide
MWLQSLLLLGTVACSIS

SEQ ID NO: 35 - protein, 19 aa, Artificial Sequence
  Human IgG signal peptide
MDWTWRILFLVAAATGAHS

SEQ ID NO: 36 - protein, 21 aa, Artificial Sequence
  CD8 signal peptide
MALPVTALLLPLALLLHAARP

SEQ ID NO: 37 - protein, 17 aa, Artificial Sequence
  Linker peptide 1
GSTSGSGKPSGEGSTKG

SEQ ID NO: 38 - protein, 5 aa, Artificial Sequence
  Linker peptide 2
GGGGS

SEQ ID NO: 39 - protein, 15 aa, Artificial Sequence
  hinge domain IgG1
EPKSCDKTHTCPPCP

SEQ ID NO: 40 - protein, 12 aa, Artificial Sequence
  hinge domain IgG4
ESKYGPPCPSCP

SEQ ID NO: 41 - protein, 62 aa, Artificial Sequence
  hinge domain CD8 alpha
ALSNSIMYFSHFVPVFLPAKPTTTPAPRPPTPAPTIASQPLSLRPEASRPAAGGAVHTRGLD

SEQ ID NO: 42 - protein, 80 aa, Artificial Sequence
  transmembraine domain CD8 alpha
AWVSACDTEDTVGHLGPWRDKDPALWCQLCLSSQHQAIERFYDKMQNAESGRGQVMSSLAELEDDFKEGYLETVAAYYEE

SEQ ID NO: 43 - protein, 29 aa, Artificial Sequence
  transmembraine domain CD28
KPFWVLVVVGGVLACYSLLVTVAFIIFWV

SEQ ID NO: 44 - protein, 41 aa, Artificial Sequence
  co-stimulatory domain CD28
RSKRSRLLHSDYMNMTPRRPGPTRKHYQPYAPPRDFAAYRS

SEQ ID NO: 45 - protein, 24 aa, Artificial Sequence
  co-stimulatory domain DAP10
LCARPRRSPAQEDGKVYINMPGRG

SEQ ID NO: 46 - protein, 42 aa, Artificial Sequence
  co-stimulatory domain CD137L
KRGRKKLLYIFKQPFMRPVQTTQEEDGCSCRFPEEEEGGCEL

SEQ ID NO: 47 - protein, 112 aa, Artificial Sequence
  primary stimulatory domain CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 560 aa, Artificial Sequence
  BCMA-CAR 1
MWQLLLPTALLLLVSAEVQLLESGGGLVQPGGSLRLSCAASGFTFSNYDMSWVRQAPGKGLEWVSWIYPSDSSIYYADSVKGRFTISRDNSKNTLYLQMNSLRAEDTAVYYCARGPFANKYRQFDYWGQGTLVTVSSGSTSGSGKPSGEGSTKGQSVLTQPPSASGTPGQRVTISCSGSSSNIGSNSVSWYQQLPGTAPKLLIYADSKRPSGVPDRFSGSKSGTSASLAISGLRSEDEADYYCGSWDYSLSGYVFGGGTKLTVLALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSGGGGSKRGRKKLLYIFKQPFMRPVQTTQEEDGCSCRFPEEEEGGCELGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 553 aa, Artificial Sequence
  BCMA-CAR 2
MWQLLLPTALLLLVSAEVQLLESGGGLVQPGGSLRLSCAASGFTFSNYGVHWVRQAPGKGLEWVSYISYSGGTYYNPSLKSRFTISRDNSKNTLYLQMNSLRAEDTAVYYCARDSDDFGFDYWGQGTLVTVSSGSTSGSGKPSGEGSTKGQSVLTQPPSASGTPGQRVTISCQGDSLRSYYVNWYQQLPGTAPKLLIYDHSKRPTGVPDRFSGSKSGTSASLAISGLRSEDEADYYCQSYDSSTVVFGGGTKLTVLGALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSGGGGSKRGRKKLLYIFKQPFMRPVQTTQEEDGCSCRFPEEEEGGCELGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 553 aa, Artificial Sequence
  BCMA-CAR 3
MWQLLLPTALLLLVSAEVQLLESGGGLVQPGGSLRLSCAASGFTFSDYGLSWVRQAPGKGLEWVSLIDSSGSSTFYADSVKGRFTISRDNSKNTLYLQMNSLRAEDTAVYYCAKEHGLFDSWGQGTLVTVSSGSTSGSGKPSGEGSTKGEIVLTQSPGTLSLSPGERATLSCKASQDIDDDINWYQQKPGQAPRLLIYDASLRATGIPDRFSGSGSGTDFTLTISRLEPEDFAVYYCQQSLRTPITFGQGTKLEIKRALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSGGGGSKRGRKKLLYIFKQPFMRPVQTTQEEDGCSCRFPEEEEGGCELGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 553 aa, Artificial Sequence
  BCMA-CAR 4
MWQLLLPTALLLLVSAEVQLLESGGGLVQPGGSLRLSCAASGFTFSGHYWSWVRQAPGKGLEWVSTVSGSGGDTFYADSVKGRFTISRDNSKNTLYLQMNSLRAEDTAVYYCARGHSVMDVWGQGTLVTVSSGSTSGSGKPSGEGSTKGEIVLTQSPGTLSLSPGERATLSCRASQGIDSYVAWYQQKPGQAPRLLIYDASLRATGIPDRFSGSGSGTDFTLTISRLEPEDFAVYYCQQYNSWPITFGQGTKLEIKRALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSGGGGSKRGRKKLLYIFKQPFMRPVQTTQEEDGCSCRFPEEEEGGCELGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR